(19) United States
(12) Patent Application Publication
(10) Pub. No.: US 2026/0174365 A1
(43) Pub. Date: Jun. 25, 2026

(54) Oocyte Retrieval Tubing System

(71) Applicant: R V Dalton Pty Ltd, Ballarat (AU)

(72) Inventor: Russell Victor Dalton, Ballarat (AU)

(21) Appl. No.: 19/125,976

(22) PCT Filed: Nov. 29, 2023
(86) PCT No.: PCT/AU2023/051228

§ 371 Date: Apr. 30, 2025

(30) Foreign Application Priority Data
Nov. 30, 2022 (AU) 2022903637

(51) Int. Cl. A61B 5/15; A61B 17/00; A61B 17/34

(52) U.S. Cl. CPC A61B 5/150656; A61B 17/3415; A61B 2017/00199

(57) ABSTRACT

An oocyte retrieval tubing system, comprising a manifold for receiving a supply of heated air; an outer tubing for accommodating an internal transfer tube extending between a collection vessel and an oocyte retrieval needle during use; the outer tubing comprising a first and second separate insulating tubes connected to each other via the manifold, wherein each of the first and second insulating tubes are configured to be lengthwise adjustable such that their operating lengths could be adjusted relative to each other to regulate the flow of heated air, received through the manifold disposed therebetween, in the outer tubing so as to maintain a stable and evenly distributed temperature within the outer tubing for oocyte retrieval.

### TECHNICAL FIELD

The invention is directed broadly towards an oocyte retrieval tubing system. In particular, there is provided a unit that comprises an insulated passageway for accommodating transfer tubing of an oocyte retrieval needle and systems and methods incorporating and/or using said unit.

### BACKGROUND

In Vitro Fertilization (IVF) is a medical procedure that is used to overcome a range of fertility issues, by which an egg and sperm are combined outside of the body.

A critical step in the IVF cycle, is the retrieval, or harvesting, of developing eggs known as oocytes. The oocytes are suspended within follicular fluid of follicles within the ovaries of a female patient. During an IVF procedure, an oocyte retrieval needled is passed through the vaginal wall and into an ovary under ultrasound guidance. The guided oocyte retrieval needle is used to puncture and enter each follicle, with the follicular fluid being drained from the follicle under induced negative pressure. The follicular fluid is communicated through the needle and along a length of transfer tubing into a collection vessel such as a test tube via a vacuum pump.

The harvested oocytes contain an intracellular structure known as a meiotic spindle. The meiotic spindle is a microtubule-based structure that facilitates the segregation chromosomes during the fertilization process. The integrity of the meiotic spindle is of upmost importance to the viability of the oocyte. The microtubules of the spindle are particularly sensitive to temperature change, in particular cooling, with studies showing that temperature changes as small as 1° C. can adversely disrupt the meiotic spindle. The greater the magnitude and/or duration of the temperature drop, the more likely the physical damage to the oocytes.

In view of the above, existing IVF processes seek to minimise temperature drop of the collected follicular fluid. For example, the collection vessel is typically seated on a heat block and warmed to a body temperature of 37° C. The oocytes are then stored in an incubator that substantially maintains the temperature of the oocytes at this desirable level.

A drawback of conventional IVF processes, however, is the lack of regulation of the temperature of the follicular fluid as it is transferred from the body to the collection vessel. Given the small diameter of the transfer tubing, there is a rapid temperature drop between the times that the oocyte containing follicular fluid is removed from the follicle to the point it reaches the collection vessel. Further, the mixing of the (cooler) follicular fluid from the transfer tubing with the (warmed) follicular fluid within the collection vessel also results in fluctuations in the temperature within the fluid, and subsequently, the oocytes upon reaching the collection vessel.

Within this context, there is a need for an improved heat exchanger or to at least provide the public with a useful choice. The present invention was conceived with these shortcomings in mind.

### SUMMARY

In a first aspect, the invention provides an oocyte retrieval tubing system, comprising a manifold for receiving a supply of heated air; an outer tubing for accommodating an internal transfer tube extending between a collection vessel and an oocyte retrieval needle during use; the outer tubing comprising a first and second separate insulating tubes connected to each other via the manifold, wherein each of the first and second insulating tubes are configured to be lengthwise adjustable such that their operating lengths could be adjusted relative to each other to regulate the flow of heated air, received through the manifold disposed there between, in the outer tubing so as to maintain a stable and evenly distributed temperature within the outer tubing for oocyte retrieval.

The manifold may comprise a body having a first end and a second end, with the first insulating member being coupled to the manifold proximate the first end and the second insulating member being coupled to the manifold proximate the second end. The body of the manifold may have a diameter that tapers from a first diameter at the first end to a reduced, second, diameter at the second end.

In some embodiments, the manifold may comprise receiving portions that extend from the first and second ends of the body, with the first and second members being respectively coupled to the manifold via the receiving portions. The receiving portions may comprise constant diameter sections that project linearly from the respective first and second ends of the body. The receiving portion of the second end may include an engagement element for securing the second insulating member to the manifold.

The passageway may be a flexible passageway. In particular, the first and second insulating members may comprise flexible conduits. The internal temperature of the passageway is selected to substantially approximate an internal body temperature of the patient from which the oocyte is being collected. Preferably, the provided internal temperature of the outer tubing is substantially maintained within a range of about 36.8° C. and 37.2° C., during use.

In some embodiments, the first and/or second insulating tubes may be of an extendable member. Each of the first and second insulating tubes may comprise a collapsible concertina type section for length adjustability. The first insulating tube may have a length that is greater than the second insulating tube. The first insulating tube may have an operating length about four times the operating length of the second insulating tube when the first insulating tube is in an extended configuration.

In some embodiments, the operating length of the first insulating tube is between about 40 and 65 cm, and the operating length of the second insulating tube is between about 10 and 25 cm. In some embodiments, the diameter of the first insulating tube is about 20 mm, and the diameter of the second insulating tube is about 15 mm.

In some embodiments, the supply of heated air is received through the manifold and flowing into the outer tubing at a flow volume of between about 30 and 35 litres per minute.

The manifold may include an opening for receiving the heated air supply, the opening being in fluid communication with the first and second ends of the body. The manifold may include a branch portion that extends from the body between the first and second ends thereof, with the opening for the heated air supply being provided by an open end of the branch portion. The branch portion may extend outwardly from the body portion in a direction of the second end.

In some embodiments, the unit may further comprise an air supply line that extends from the opening of the manifold to a heat source. The air supply line may have a diameter that is smaller than that of the first and second insulating tubes. A connector may be disposed at a free end of the air supply line that is configured to connect to the heat source. The heated air may be supplied at a temperature of between 37.0° C. and 37.4° C.

In a second aspect, the invention provides an oocyte retrieval system, comprising: an oocyte retrieval needle; transfer tubing that is attached to the retrieval needle and extends from the needle to a collection vessel; and an oocyte retrieval tubing system as described above.

In a third aspect, the invention provides a method of retrieving oocyte, comprising the steps of: communicating heated air into a passageway to maintain a selected internal temperature within the passageway; and transferring follicular fluid from an oocyte retrieval needle and along a transfer tube that is accommodated within the passageway to a collection vessel; wherein the internal temperature of the passageway is selected to maintain a temperature of the follicular fluid within the transfer tubing.

### BRIEF DESCRIPTION OF THE DRAWINGS

The invention will now be described, by way of non-limiting example only, with reference to the accompanying drawings, in which:

FIG. 1 is a perspective view of a temperature regulating unit according to an embodiment of the invention, illustrating the unit in a preparatory, retracted configuration;

FIG. 2 is a perspective view of the temperature regulating unit of FIG. 1, illustrating the unit in an operating, extended configuration;

FIGS. 3 to 5 illustrate a manifold of the temperature regulating unit of FIG. 1;

FIG. 6 is a schematic representation of an oocyte retrieval system that includes a temperature regulating unit according to an embodiment of the invention; and

FIG. 7 is a flowchart schematically illustrating an oocyte retrieval method that includes the use of a temperature regulating unit according to an embodiment of the invention.

### DETAILED DESCRIPTION

In the following detailed description, reference is made to accompanying drawings which form a part of the detailed description. It will be readily understood that the aspects of the present disclosure, as generally described herein and illustrated in the drawings may be arranged, substituted, combined, separated and designed in a wide variety of different configurations, all of which are contemplated in this disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the example methods and materials are described herein.

In general terms, the oocyte retrieval tubing system shown in the Figures is an apparatus comprising an insulated and actively warmed passageway that is adapted to accommodate the transfer tubing of an oocyte retrieval needle. In particular, the passageway includes an opening that is adapted to receive a supply of heated air from an external heat source. The heated air is supplied into the passageway at a temperature substantially approximating an internal body temperature of a patient from which follicular fluid and oocytes contained therein are to be collected. In use, with the passageway extending substantially along a length of the transfer tubing, temperature change in follicular fluid is avoided or at least reduced as the collected fluid is transferred from the patient to a collection vessel.

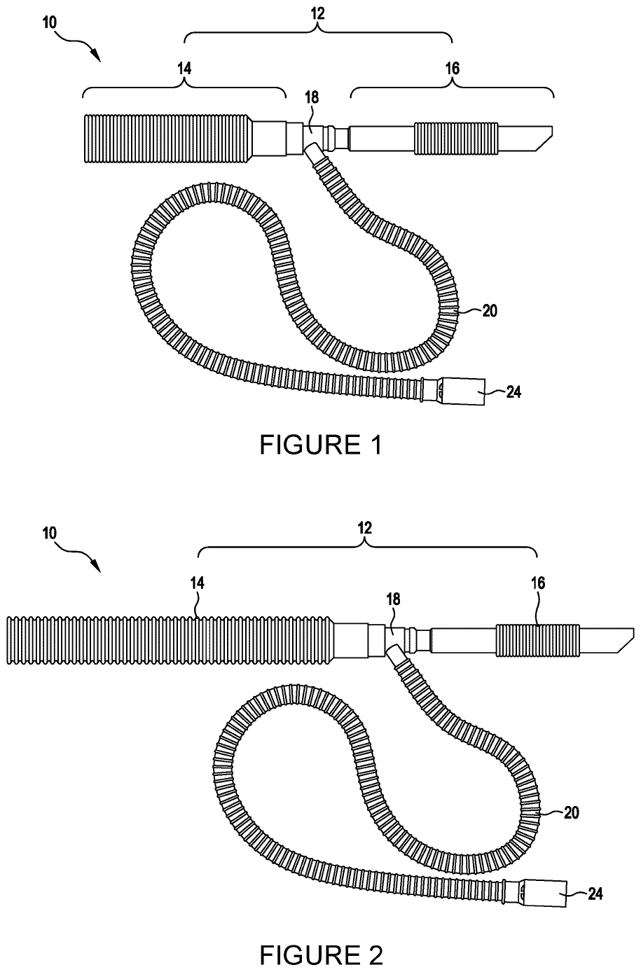

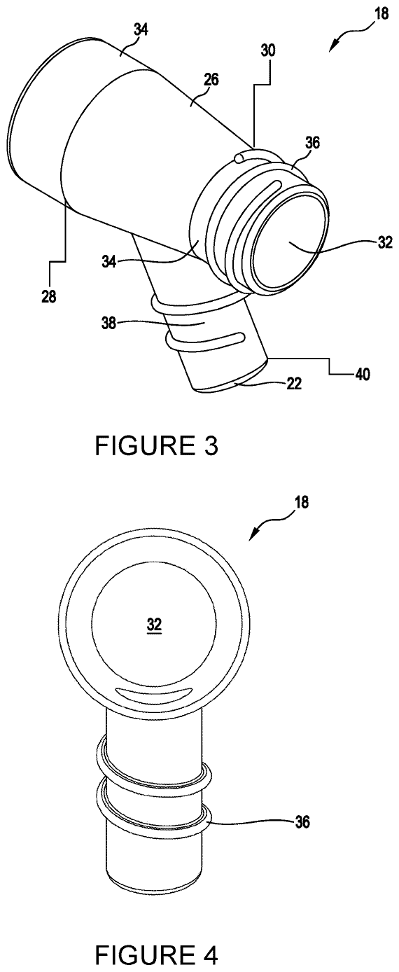

With particular reference to FIGS. 1 and 2, an embodiment of the invention in the form of a temperature regulating unit 10 that comprises a passageway 12 (outer tubing) that is adapted to receive an internal transfer tubing extending between a collection vessel and an oocyte retrieval needle. As shown, the passageway is formed, at least in part, by a first insulating member 14 (tube) and a second insulating member 16 (tube) that is fluidly connected thereto. Each of the insulating members 14, 16 are respectively coupled to a manifold 18. The manifold 18 provides fluid communication between the first and second insulating members 14, 16. The manifold 18 will be described in more detail later with particular reference to FIGS. 3 to 5.

The insulating members 14, 16 are elongate conduits, having a diameter suitable for accommodating standard transfer tubing associated with oocyte retrieval needles. The flexible conduits are preferable formed of soft, highly flexible material. Examples of suitable materials include polymeric materials such a polyethylene and polypropylene. Preferably, the second member 16 is coil reinforced.

The lengths and/or diameters of the first and second members 14, 16 are chosen to ensure a substantially even and consistent heat distribution and flow of the heated air supply across the passageway 12. Preferably, the first and/or second members 14, 16 are configured to be lengthwise adjustable such that their operating lengths could be adjusted relative to each other to regulate the flow of heated air inside the passageway 12. Specifically, the operating lengths and diameters of the members 14, 16 are calculated in accordance with the Hagen-Poiseuille law, based on the desired complete length of the passageway 12—with said complete length being approximately equal to the length of the transfer tubing extending between the oocyte retrieval needle and the collecting vessel. Typically, the length of the transfer tubing can be up to 75 cm. With particular reference to the illustrated embodiments, the first insulating member 14 has an inside diameter of about 20 mm and the second insulating member 16 has an inside diameter of about 15 mm. Such diameters also facilitate simple and easy insertion and maneuvering of the oocyte retrieval needle and connected transfer tubing through the respective members 14, 16. Furthermore, the second insulating member 16, as shown, has an operating length of between about 10 cm and 25 cm. Preferably, the ratio of a length of the first member 14 to the second member 16 is about 4 to 1. The applicant has found that the ratio of 4:1 is provides an optimal location of the manifold within passageway 12, so as to minimize the ergonomic impact on the actions of a surgeon, and/or a nurse during changeover and/or attachment of the collection tubing to the collecting vessel during the IVF procedure. It is understood, however, that the lengths and/or diameters of the first and second members 14, 16 can vary based on the required length of the passageway 12 and/or required minimum internal diameters, according to the Hagen-Poiseuille law. It is to be appreciated that the dual insulating tubes and their respective operating lengths and diameters are designed to handle heated air flowing through the manifold and into the passageway 12 at a flow volume of between about 30 and 35 litres per minute.

The adjustable lengths of the first member 14 and the second member 16 can make it easier to insert and feed the internal transfer tube as well as the oocyte retrieval needle through the passageway 12. As shown in one embodiment, the first member 14 and second member 16 are configured so that part of their lengths are provided as a collapsible concertina type conduit. FIG. 1 shows the first member 14 in a collapsed or retracted state that represents a preparatory configuration of the temperature regulating unit 10 associated with preparatory steps of an oocyte retrieval process. FIG. 2 shows the first member 14 in an extended state that represents an operating configuration of the unit 10 that is associated with operating steps of the oocyte retrieval process. In the extended state, the first member 14 has an operating length of between about 40 cm and 65 cm. With the first member 14 in the extended state, the passageway 12 extends substantially along a complete length of the transfer tubing. An additional advantage of the first member 14 and/or the second member 16 having a section that is collapsible or lengthwise adjustable is that the process of inserting the internal transfer tubing and oocyte retrieval needle through the passageway 12 is made easier when the members 14, 16 are collapsed. The lengths of the members 14, 16 are then extended to the correct relative lengths once the internal transfer tube is inserted and the system is ready for use. Additionally, when in the collapsed or retracted configuration, the first member 14 and second member 16 provide a smaller more compact unit 10 to improve the handlebility of said unit 10 during the preparation for oocyte collection.

The temperature regulating unit 10 also includes an air supply line 20. The air supply line 20 is adapted to communicate warmed air from a heat source (not shown) into the passageway 12. Specifically, the air supply line 20 is coupled to the manifold 18, communicating air through an opening 22 thereof. The diameter of the air supply line 20 can affect the flow-rate of the heated air into the passageway 12. As shown, the air supply line 20 is provided as a flexible conduit having an internal diameter of about 10 mm and a length of about 30 cm to 60 cm. Preferably, the warmed air has a temperature of about 37° C. and 37.4° C. Experimental trials by the inventor have demonstrated that the supply of warmed air within this range results in an internal temperature within the passageway 12 of between 36.8° C. and 37.2° C. Providing an internal passageway temperature within the range is particularly desirable as it closely approximates the average internal body temperature within the follicular fluid of a human being.

A connector 24 is provided at a free end of the air supply line 20. The free end is understood to be the end of the air supply line 20 that is not coupled to the manifold 18. The connector 24 is configured to fluidly connect the air supply line 20 to an outlet of the external heat source. Preferable the connector 24 is a “quick lock” type connector that provides simple connection and/or removal of the air supply line 20 with the heat source. As shown, the connector 24 is a “universal” type connector suitable for connecting with different types of heat source that are typically used and widely available in IVF clinics and/or surgical settings. In other embodiments, the connector 24 may be a removable connector that is provided as one of several connectors within a kit, with each connector within the kit being selectably attachable to the free end of the air supply line 20 to suit a particular heat source. The heat source may include controls which enable a user of the regulating unit 10 to select the internal or substantially regulate the internal temperature within the passageway 12. For example, the heat source may have controls to regulate the flow rate of the heated air or the supplied temperature thereof. Alternatively, such controls may be provided as part of the regulating unit 10 itself, with the unit 10 being configured to interact or otherwise control the heat source via the connector 24.

The manifold 18 will now be described with particular reference to FIGS. 3 to 5.

Best shown in FIG. 3, the manifold 18 comprises a cylindrical body 26 that extends substantially linearly from a first end 28 to a second end 30. The body 26 is a hollow body, with a central bore 32 extending between the first end 28 and the second end 30. The bore 28 forms part of the passageway 12. The body 26 has a convergent profile, with an external diameter that tapers from a first outer diameter at the first end 28 to a reduced second outer diameter at the second end 30. With particular reference to the illustrated embodiment, the manifold has a length of about 2 cm, tapering from a first outer diameter of about 20 mm to a second outer diameter of about 15 mm. The wall thickness of the cylindrical body is about 1 mm, such that an inner diameter of the body tapers from about 18 mm to about 13 mm from the first end 28 to the second end 30.

The manifold 18 also includes a pair of receiving portions 34, which project outwardly from the respective first and second ends 28, 30. The receiving portions 34 are configured to engage or otherwise couple with the insulating members 14, 16. As shown in the Figures, the receiving portions 32 are comprise constant diameter sections that project outwardly from the first and second ends 28, 30. Specifically, the receiving portions 34 have a length of about 10 mm, such that a complete axial length of the manifold 18 is about 40 mm. An engagement element 34 in the form of a screw thread is disposed within the receiving portion 34 of the second end 30. The thread is a raised thread that allows twisting attachment of the second member 16. It is understood that other forms of engagement element 36 and/or other standard means to secure insulating members 14, 16 to the manifold 18 are also contemplated. For example, the receiving portions 34 may, alternatively, be provided as push—in type fittings configured to respectively receive the first and second members 14, 16 therein.

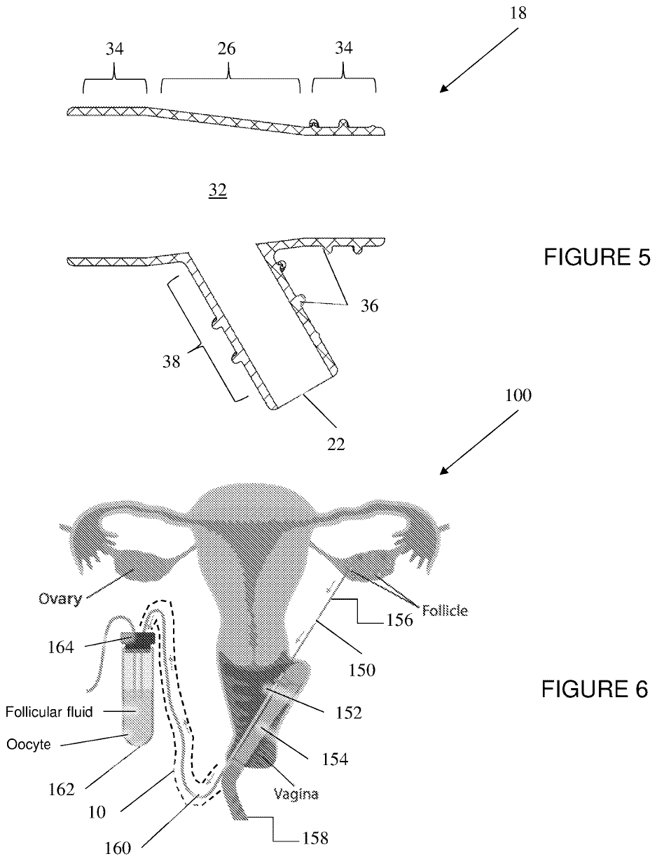

Turning now to FIGS. 4 and 5. The manifold 18 also comprises a branch portion 38. The branch portion 38 extends outwardly from the cylindrical body 26 to a free or distal end 40 that provides the opening 22. As shown, the branch portion 38 is a tubular limb that extends for a length of about 10 mm substantially linearly in a direction towards the second end 30 of the body 26. The branch portion 38 is provided at an angle of approximately 60 degrees from a longitudinal axis of the passageway 12. With respect to the illustrated embodiment, the branch portion 38 as an inner diameter of about 8 mm and an outer diameter of about 10 mm. It is understood that the length and inner diameter of the branch portion 38 is also selected in accordance with the desired flow characteristics of the heater air supply from air supply line 20. Engagement elements 36 are provided along the branch portion 38 and in particular proximate the distal end 40, to facilitate attachment of the air supply line 20 thereto. It is understood that other forms of the engagement element 36 and/or other standard means to secure air supply line 20 to branch portion 38 are also contemplated.

Best shown in FIG. 5, the branch portion 38 is fluidly connected to the bore 28 of the body 26. What is meant by this is that the flow of heater air from the air supply line 20 is transferred through the branch portion 38 into the bore 28 of the manifold 18. Due to the angle at which the branch portion meets the bore 28, the flow of heated air is directed towards the first end 28 and the first member 14 connected thereto. It is understood that backflow of the heated air results in said air circulating along the passageway 12. Specifically, the angle of direction of branch portion 38 towards the first end 28 creates a preferential flow of warm air towards the longer, more voluminous part of the passageway 12, and maintains a reservoir of warm air therein. This allows for consistent flow in this direction as determined by the Hagen-Poiseuille law if the length of 14 is in the extended configuration. In addition, the angle of branch portion 38 also provides for better ergonomic function of the device 10 during the oocyte collection procedure.

The manifold 18 is preferably integrally formed in a moulding operation. What is meant by this is that the branch portion 28 and the cylindrical body 26 form a unitary body. Suitable polymeric materials include PTFE, nylon and polypropylene. PTFE is particularly preferred due to its low friction characteristics which assist in the fitment and subsequent removal of first and second insulating members 14, 16 and the air supply line 20. Further, it is understood that the material selection for the manifold 18 and insulating members 14, 16 is chosen to allow for sterilization of the unit 10 via gamma irradiation.

The unit 10 may form part of an oocyte retrieval system 100. The oocyte retrieval system 100 will now be described with reference to FIG. 6.

Oocyte retrieval system 100 comprises an oocyte retrieval needle 150 that is configured to harvest or collect oocyte from the ovaries of the female patient. The oocyte retrieval needle 150 is preferably a single lumen needle although double lumen needles are also contemplated. The needle 150 is adapted to be introduced into the vagina with the assistance of a needle guide 152. The needle guide 152 may be attached or otherwise coupled to an ultrasound probe 154. The ultrasound probe 154 is used by a surgeon to assist in the locating and identifying of follicles that are to be drained. A distal end 156 of the needle 150 is configured to pierce the vaginal wall and enter the follicles. At a proximal end 158 of the needle that may include a handle for the surgeon, flexible transfer tubing 160 is connected. The transfer tubing 160 is adapted to aspirate follicular fluid (within which the oocyte is suspended) from the follicle and transfer it to a collection vessel 162, as indicated by the directional arrows in the Figure. As shown, the collection vessel 162 is a test tube. The test tube 162 is preferably seated upon a heating pad or similar heating device to hold the collected fluid at a temperature substantially equivalent to an internal body temperature of the subject. For human patients, the internal body temperature is about 37° C. Alternatively, the test tube 162 may be heated or warmed by other means. For example, the test tube 162 may be accommodated within a heated container or wrapped in a heated jacket.

A temperature regulating unit 10 as described herein is fitted around the transfer tubing 160. In the Figure, the passageway 12 of the unit 10 is schematically represented in dashed outline for clarity. The passageway 12 extends substantially along a complete length of the transfer tubing 160, between the proximal end 158 of the needle 150 and the collection vessel 162. The internal volume of the passageway 12 provides an insulated, warmed, humidified environment that eliminates or at least substantially reduces temperature drop in the follicular fluid as it is communicated along the transfer tubing 160. The second member 16 of the unit 10 may be configured to have an open end located above, or proximate to a silicone stopper 164 that provides a lid of the collection vessel 162 to maintain a substantially localised and warmed humidified environment. The open end of the second member 16 may include a contraction adapted to focus the exhaust of the warmed air away from the passageway 12. In other embodiments, the second member 16 may directly couple to the stopper 164. Further, the first member 14 of the unit may be configured to have an open end located above or proximate to the distal end of the needle 150. The open end of the first member 14 may include a contraction adapted to focus the exhaust of the warmed air away from the passageway 12. The first member may, for example, extend along a partial length of a handle portion of the needle 150, with the handle portion comprising a part of the needle 150 which is outside of the needle guide 152. In other embodiments, the first member may directly couple to the distal end of the needle 150. Advantageously, the flexibility of the passageway 12 of the unit 10 ensures that there is minimal if any undesirable ergonomic impact or hindrance in the maneuverability of the needle 150 and associated equipment used by a surgeon and/or scrub nurse during the oocyte retrieval procedure.

As described herein, the temperature regulating unit 10 is a separate apparatus that is distinct from the needle 150 and transfer tubing 160 associated therewith. A benefit of this is that the temperature regulating unit 10 can be retrofitted to and/or incorporated with existing surgical equipment that is typically used within IVF settings. In this way, changes and or disruptions to existing/standard workflows of surgeons performing oocyte retrieval procedures are minimized. Accordingly, the temperature regulating unit 10 thereby works to improve the success rate of oocyte collection without complicating and/or introducing unnecessary drawbacks into well established procedures and methodologies. It is understood, however, that in other embodiments, the temperature regulating unit 10 could be integrated with and/or form part of other pieces of surgical equipment used for oocyte retrieval. For example. The temperature regulating unit 10 may be provided with an integrated internal channel that substantially serves as the transfer tubing, with the channel extending along the passageway and being adapted to receive follicle fluid from the needle 150, and communicate said fluid to the collection vessel 160.

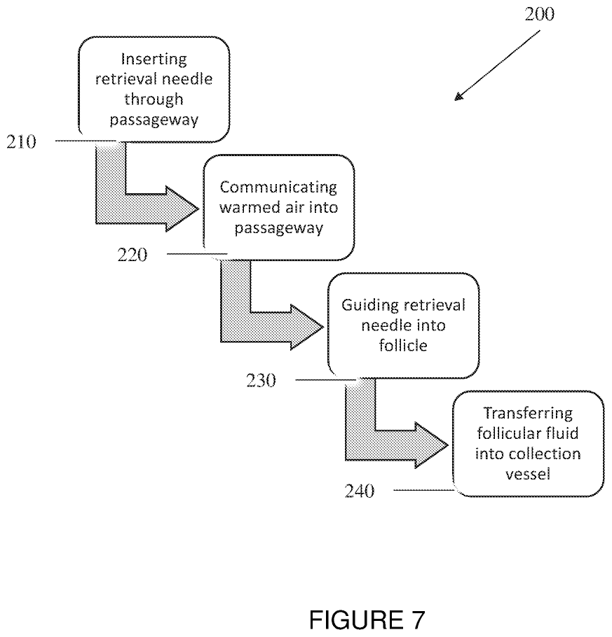

A method 200 of retrieving oocyte from a female patient using the system 100 as described herein as part of an IVF procedure will now be described with reference to FIG. 7.

In a preparatory insertion step 210, the oocyte retrieval needle 150 and connected transfer tubing 160 are inserted into the temperature regulating unit 10 via the second member 16 thereof. The needle 150 and tubing 160 is then passed along the passageway 12, such that the needle 150 projects therefrom. During the insertion of the needle 150, the temperature regulating unit is in the preparatory configuration with the extendable first member 14 being in the collapsed or retracted state. Once the needle 150 has been passed through the passageway 12, the first member 14 is extended, such that the passageway 12 extends along a substantial length of the connected transfer tubing 160.

In a subsequent warming step 220, warmed air is pumped or otherwise communicated into the passageway 12. Preferably, the air supply line 20 is first connected to the opening 22 of the manifold 18, before the flow of warmed air from the external supply is initiated. The warmed air is supplied at a temperature substantially equivalent to an internal body temperature of the female patient, for example 37° Celsius. As the warmed air is pumped through the opening 22 and into the manifold 18, the air flow is substantially evenly distributed through the first and second ends 28, 30 and into the connected insulating members 14, 16. The warmed air supply creates a warm and substantially stable constant temperature air jacket within the passageway 12, with the air jacket surrounding the transfer tubing 160. Temperature and/or pressure sensors (not shown) may be disposed within the passageway 12, for monitoring the internal environment of the passageway 12. The sensors may be in communication with a controller (not shown) of the external heat source, to adapt the flow characteristics in order to obtain the desired environmental conditions within the passageway 12.

With the environmental conditions within the passageway 12 having reached a desired steady-state level, the oocyte retrieval process continues as per typical practice. Specifically, in a collection step 230, the retrieval needle 150 is inserted into and guided along the needle guide 152 into the selected follicle. Preferably, the needle 150 is guided into position with the assistance of ultrasound probe 154 which is adapted to carry the needle guide 152.

Once the tip or distal end 156 of the needle 150 has pierced the follicle, aspiration begins, with vacuum pressure applied by an external vacuum source being applied to drain follicular fluid from the follicle and through the needle 150 and into the transfer tubing 160. During a transfer step 240, the follicular fluid is than drawn along said transfer tubing 160-accommodated within the passageway 12—and into the collection vessel 162. Advantageously, with the internal environment of the passageway 12 substantially surrounding the transfer tubing 160, there is little to no temperature drop within the follicular fluid during this transfer step 240.

Summarily, it is to be understood that the temperature regulating unit as described herein may advantageously improve the viability of oocyte that is retrieved from a female patient as part of an IVF process. In particular, the temperature regulating unit providing a substantially stable, constant temperature environment within a passageway that substantially envelopes the transfer tubing that is used to communicate said fluid from the oocyte retrieval needle to the collecting vessel. In this way, temperature drop within the follicular fluid is substantially avoided or at least minimized, thereby reducing the possibility of damage to the meiotic spindle within with oocyte which can negatively affect the likelihood of said oocyte being used to form a viable embryo.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Throughout this specification and the claims which follow, unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps.

REFERENCE NUMERALS

| | |
|---|---|
| 10 | Oocyte retrieval tubing system |
| 12 | Passageway (outer tubing) |
| 14 | First member (first insulating tube) |
| 16 | Second member (second insulating tube) |
| 18 | Manifold |
| 20 | Air supply line |
| 22 | Opening |
| 24 | Connector |
| 26 | Cylindrical body |
| 28 | First end |
| 30 | Second end |
| 32 | Bore |
| 34 | Receiving portion(s) |
| 36 | Engagement element(s) |
| 38 | Branch portion |
| 40 | Free end |
| 100 | Oocyte retrieval system |
| 150 | Oocyte retrieval needle |
| 152 | Needle guide |
| 154 | Ultrasound probe |
| 156 | Tip end |
| 158 | Handle end |
| 160 | Transfer tubing |
| 162 | Collection vessel |
| 164 | Stopper |
| 200 | Method of retrieving oocyte |
| 210 | Insertion step |
| 220 | Warming step |
| 230 | Guiding step |
| 240 | Transferring step |

1. An oocyte retrieval tubing system, comprising
a manifold for receiving a supply of heated air;
an outer tubing for accommodating an internal transfer tube extending between a collection vessel and an oocyte retrieval needle during use;
the outer tubing comprising a first and second separate insulating tubes connected to each other via the manifold, wherein each of the first and second insulating tubes are configured to be lengthwise adjustable such that their operating lengths could be adjusted relative to each other to regulate the flow of heated air, received through the manifold disposed therebetween, in the outer tubing so as to maintain a stable and evenly distributed temperature within the outer tubing for oocyte retrieval.

2. The oocyte retrieval tubing system of claim 1, wherein the internal temperature of the outer tubing is substantially maintained within a range of about 36.8° C. and 37.2° C., during use.

3. The oocyte retrieval tubing system of claim 1, wherein the manifold comprises a body having a first end and a second end, with the first insulating tube being coupled to the manifold proximate the first end and the second insulating tube being coupled to the manifold proximate the second end.

4. The oocyte retrieval tubing system of claim 3, wherein the body of the manifold has a diameter that tapers from a first diameter at the first end to a reduced, second, diameter at the second end.

5. The oocyte retrieval tubing system of claim 3, wherein the manifold comprises receiving portions that extend from the first and second ends of the body, with the first and second tubes being respectively coupled to the manifold via the receiving portions.

6. The oocyte retrieval tubing system of claim 5, wherein the receiving portions comprise constant diameter sections that project linearly from the respective first and second ends of the body.

7. The oocyte retrieval tubing system of claim 1, wherein each of the first and second insulating tubes is provided with a collapsible concertina type section for length adjustability.

8. The oocyte retrieval tubing system of claim 1, wherein the first insulating tube has a maximum length that is greater than a maximum length of the second insulating tube.

9. The oocyte retrieval tubing system of claim 8, wherein the first insulating tube has an operating length of about four times the operating length of the second insulating tube.

10. The oocyte retrieval tubing system of claim 1, wherein the operating length of the first insulating tube is between about 40 and 65 cm.

11. The oocyte retrieval tubing system of claim 1, wherein the operating length of the second insulating tube is between about 10 and 25 cm.

12. The oocyte retrieval tubing system of claim 1, wherein the diameter of the first insulating tube is about 20 mm.

13. The oocyte retrieval tubing system of claim 1, wherein the diameter of the second insulating tube is about 15 mm.

14. The oocyte retrieval tubing system of claim 3, wherein the manifold includes an opening for receiving the heated air supply, the opening being in fluid communication with the first and second ends of the body.

15. The oocyte retrieval tubing system of claim 14, wherein the manifold includes a branch portion that extends from the body between the first and second ends thereof, with the opening for the heated air supply being provided by an open end of the branch portion.

16. The oocyte retrieval tubing system of claim 15, wherein the branch portion extends outwardly from the body portion in a direction of the second end.

17. The oocyte retrieval tubing system of claim 14, further comprising an air supply line that extends from the opening of the manifold to a heat source.

18. The oocyte retrieval tubing system of claim 17, wherein the air supply line has a diameter that is smaller than that of the first and second insulating tubes.

19. The oocyte retrieval tubing system of claim 10, further comprising a connector disposed at a free end of the air supply line that is configured to connect to the heat source.

20. The oocyte retrieval tubing system of claim 1, wherein the supply of heated air is received through the manifold and flowing into the outer tubing at a flow volume of between about 30 and 35 litres per minute.

21. An oocyte retrieval system, comprising:
an oocyte retrieval needle;
transfer tubing that is attached to the retrieval needle and extends from the needle to a collection vessel; and
an oocyte retrieval tubing system according to claim 1.
